(19) United States
(12) Patent Application Publication
(10) Pub. No.: US 2016/0090571 A1
(43) Pub. Date: Mar. 31, 2016

(54) CULTURE MEDIA AND METHODS FOR CULTURING MICROORGANISMS

(71) Applicant: Orior, LLC, Roseland, NJ (US)

(72) Inventor: Clifford E. Carnicom, Wallace, ID (US)

(73) Assignee: Orior, LLC, Roseland, NJ (US)

(21) Appl. No.: 14/871,544

(22) Filed: Sep. 30, 2015

Related U.S. Application Data
(60) Provisional application No. 62/057,712, filed on Sep. 30, 2014.

(51) Int. Cl. C12N 1/20; A01N 43/08; A01N 37/36; A01N 37/44

(52) U.S. Cl. CPC C12N 1/20; A01N 37/44; A01N 43/08; A01N 37/36

(57) ABSTRACT

Methods and culture media for culturing a microorganism associated with Morgellons disease are provided. In exemplary embodiments, methods are provided that may include adding a microorganism associated with Morgellons disease to a solution containing ferrous iron, sugar and water or to a solid growth medium containing agar and ferrous iron. Also provided are compositions containing a microorganism associated with Morgellons disease as well as methods for isolating lipids and proteins from this microorganism and methods for inhibiting its growth.

### CROSS-REFERENCE TO RELATED APPLICATIONS

The present application claims priority to U.S. Provisional Patent Application Ser. No. 62/057,712 entitled “Methods of Isolating Culturing and Preserving of a Novel Microorganism and Methods of Extracting Lipids and Proteins Therefrom,” filed Sep. 30, 2014, the disclosure of which is incorporated herein by reference in its entirety, as if fully set forth herein.

### BACKGROUND

1. Field of the Invention

Embodiments of the present invention are generally related to methods of isolating, culturing and preserving a microorganism associated with Morgellons disease or condition. More specifically, embodiments of the present invention relate to culturing a microorganism by adding the microorganism to a culture consistent with the present disclosure as well as methods for isolating lipids and/or proteins from this microorganism.

2. Description of the Related Art

A phenomenon most commonly referred to as “Morgellons” disease is a condition characterized by a range of various dermatologic and neuropsychiatric symptoms. These symptoms may include disfiguring sores and crawling sensations on and under the skin. Some individuals who have the disease describe unusual symptoms like strange fibers poking through their skin, or the like. The skin sores and lesions most closely resemble insect or spider bites that are worsened by scratching. The disease can feature fibers or solid materials emerging from the sores. Morgellons is also characterized by joint arthralgia, fatigue, sleep disturbances, changes in vision, mental confusion and memory loss. Morgellons can cause extreme discomfort; in severe cases, it can lead to anxiety, depression and suicide.

There is no known cure for Morgellons disease. Efforts in research and understanding in the origins and pathology of Morgellons disease are ongoing. The etiology of Morgellons is highly controversial. The scientific and medical communities have yet to confirm a causative agent. It is currently unknown whether and which infectious agents cause Morgellons, in particular because the disease resembles and is often confused with delusional parasitosis.

Aside from fibers and solid materials, the sores from Morgellons disease have been associated with visible filamentous structures that consist of proteins and lipids. The primary form of growth of these filamentous structures is an encapsulating filament sheath, which is believed to consist primarily of keratin or other substances, and it may be similar to fungal growth. The internals of the sheath consists of a sub-micron structure that is characterized at times as a spirochete/bacterial-like or chlamydia-like structure, and is termed as a Cross-Domain Bacteria (CDB) or Cross Domain Organism. Cross-Domain Bacteria (“CDB”) or Cross-Domain Organism is being classified as the most primitive form of growth from the filamentous structures. To further understand the pathology of Morgellons disease, it is advantageous to study these microorganisms in a more purified state. However, despite the tremendous research efforts, methods of isolating, culturing and preserving these microorganisms or parts thereof in a highly pure state are still in development.

Thus, there is a need for methods to isolate, culture and preserve these novel microorganisms or parts thereof in connection with Morgellons disease in a highly pure state. In addition to Morgellons disease, culture media and methods in accordance with embodiments of the present disclosure may also be used to cure or treat various other medical conditions as well.

### SUMMARY

Embodiments of the present disclosure generally relate to culture media and methods for culturing microorganisms, microorganism compositions thereof, methods for isolating lipids and/or proteins from the microorganisms, and methods for inhibiting the microorganisms.

In accordance with embodiments of the present disclosure, methods are provided for culturing a microorganism associated with Morgellons disease, or the like. Some methods in accordance with embodiments of the present disclosure may include adding the microorganism to a solution comprising ferrous iron, sugar and water, or the like. In embodiments of the present disclosure, a composition is disclosed that may include a microorganism associated with Morgellons disease in a solution comprising ferrous iron, sugar and water, or the like.

In some embodiments of the present disclosure, a method for culturing a microorganism associated with Morgellons disease may be provided. A method consistent with the present disclosure may include adding the microorganism to a solid growth medium comprising agar and ferrous iron. In embodiments of the present disclosure, a composition may be provided that includes a microorganism associated with Morgellons disease in a solid growth medium comprising agar and ferrous iron, or the like.

In some embodiments of the present disclosure, methods for chemically separating proteins and/or lipids from a microorganism associated with Morgellons disease may be provided.

In some embodiments of the present disclosure, methods for inhibiting growth of a microorganism associated with Morgellons disease may be provided.

### DETAILED DESCRIPTION

It is understood that the embodiments of the present invention are not limited to the particular methodologies, protocols and the like, described herein as they may vary. It is also to be understood that the terminology used herein is used for the purpose of describing particular embodiments only and not intended to limit the scope of the present invention. Unless defined otherwise, all technical and scientific terms used herein have the same meaning as commonly understood by one of ordinary skilled in the art to which this invention belongs.

Although treatment of Morgellons disease, or the like, is discussed throughout, the culture media and methods described herein may be used to treat various other conditions as well. The culture media and methods herein are not intended to be limited for the purpose of treating Morgellons disease.

As utilized herein, the term “microorganism” is intended to be inclusive of any single cell or multicellular microscopic organism. In particular, the term microorganism shall include all of the prokaryotes, namely the bacteria and archaea; and various forms of eukaryotes, comprising the protozoa, fungi, algae, microscopic plants (e.g., green algae), and animals such as rotifers, and planarians. Further, this term may extend to other parasites including nematodes. This term is also meant to be inclusive of Cross-Domain Bacteria (CDB) or Cross Domain Organism.

Morgellons disease or condition, as used herein, is an unexplained disorder that may be characterized by disfiguring sores and crawling sensations on and under the skin. Morgellons disease may feature fibers or solid materials emerging from these sores. Sufferers may have skin lesions that closely resemble insect or spider bites that are made worse by scratching. The most commonly affected sites are the forearms, back, chest, face, scalp and lower legs. Some of the lesions may be infected by germs commonly found on the skin, but these infections may not be the cause of the lesions. Researchers at the United States Centers for Disease Control (“CDC”) studied samples of skin, blood, urine and hair, but found no evidence that Morgellons disease is caused by an infectious agent or a substance in the environment. Researchers have been unable to determine whether Morgellons disease is a new disorder or a form of delusional parasitosis. Embodiments of the present invention may include methods of isolating, culturing and preserving the microorganism for the purpose of treating or curing Morgellons disease, or the like.

In accordance with exemplary embodiments herein, a microorganism (that may be referred to as the “Orior complex”) which may be associated with Morgellons disease has been isolated and characterized. By “associated with Morgellons disease or condition”, as used herein, it is meant that this microorganism may comprise successive and visible growth forms, especially those of a filamentous nature. The most primitive form of growth of this microorganism is sub-micron, coccus and bacterial-like. The most primitive form of growth may be termed as a Cross-Domain Bacteria (“CDB”) due to its peculiar growth patterns that appear to cross between the traditional domains of biology, namely, the Archaea, the Bacteria and the Eukarya or ecdysozoa.

The general nature of the microorganism in the early stage of growth appears to parallel that of ferrous iron oxidizing acidophile Gram-negative filament producing bacteria. It may be the smallest unit of this organism that is capable of growth and reproduction. The general size of the microorganism ranges between 0.5 and 1.0 microns, or an average of 0.6-0.8 microns. As it grows its morphology changes to more filamentous and at times spirochetal in structure. Through successful growth from the microorganism, the coccus formed structure was observed to have proteins and lipids present.

Lipids and proteins present in the microorganism were extracted using methods disclosed herein. In order to extract lipid and protein, the microorganism was first isolated, cultured and preserved in a purified state. Methods to isolate and culture this microorganism in a highly purified state in liquid or solid media, which allow for long term inert storage of the organism are disclosed herein. The culture process can be scalable and large quantities of the purified organism in the primitive state can be grown if required. Methods to chemically isolate and extract lipids and protein from the cultured microorganism are also disclosed herein.

This microorganism is believed to be widely distributed within the general environment, and has been identified and isolated in a variety of samples. These samples include, for example, human biological samples (skin filaments, blood, urine, oral filaments, biofilm production), animal organs, cultivated plants, food sources and other environmental samples. The microorganism can be isolated by microscopic examination of physical samples. The magnification level required to reliably determine the existence of the organism is approximately 3000× to 5000×. A confirmation of the identification of the organism can be made with infrared spectroscopy analysis of physical samples and cultures that are developed from the organism.

A qualitative and analytical analysis of certain lipids that have been extracted from the microorganism has been completed. Lipids are a primary biological molecule within any living organism. Several major characteristics have been identified and the results bring to the forefront additional unusual properties of the microorganism with respect to its association with Morgellons disease or condition. Some characteristics or factors that have been identified in the course of studies conducted include: (1) lipids from the microorganism appear to be highly non-polar in nature; (2) the lipids have a relatively high index of refraction; (3) the lipids appear to be composed, in the main, from long chain poly-unsaturated fatty acids; (4) the lipids appear to support combustion (i.e., oxidation) with ease; (5) the lipids appear to react readily with the halogens, such as iodine; (6) the visible light spectrum of the lipid-iodine reaction is unique and it serves as an additional means of identification and peak absorbance of the reaction is at approximately 498 nanometers; (7) a significant portion of the extracted lipids is expected to originate from the membranes of the microorganism; and (8) endotoxins within the microorganism may exist, to name a few.

Polarity can be a defining property of a molecular structure, and it is a measure of the distribution of charges within a molecule. Non-polar molecules are generally symmetric in their nature with a tendency toward an equal and symmetric distribution of charges. Polar molecules, in contrast, are usually of an asymmetric nature with the charges on the molecule unevenly distributed. Information on polarity, therefore, provides some generalized nature as to the form or nature of the molecule or substance under study.

Fatty acids are a dominant component of many lipids. They are comprised of a carboxyl group that is attached to a hydrocarbon chain. The length of this chain can vary depending upon the particular fatty acid that is involved. The carboxyl group is polar in nature and therefore the charge distribution on that particular functional group is asymmetric. The carboxyl group is also acidic in nature and this is the origin of the name of fatty acids that is attached to this common lipid structure.

The hydrocarbon chain that is attached to the carboxyl group is generally of a non-polar nature, and it serves to counteract the polar effect from the carboxyl group. Therefore, the more non-polar the lipid is, the more likely it is that the hydrocarbon is of relative greater length. A very long hydrocarbon chain (non-polar) will tend to dominate the character of the molecule in this case and ultimately make the molecule less polar.

This relationship between the polarity of and the length of the attached hydrocarbon chain provides an interpretation as to the structure of the lipid molecule. Some lipids are more or less polar than others; a highly polar lipid is indicative of lengthy hydrocarbon chains within the fatty acid. The longer the fatty acid is, the more complex the lipid structure or interactions with other molecules is likely to be. The structure of any molecule is of importance, as structure may determine function.

In polarity studies of the microorganism, lipids were mixed with a mildly polar solvent in a tube. A clear separation remained after settling. In contrast, the lipids dissolved much more readily in a highly polar solution. The specific conclusion in this case may be a lipid form that contains somewhat extensive hydrocarbon chains.

An index of refraction is a measure of the ability of a substance to bend a light wave that passes through it. It is also a measure of the speed of light though that same material. It is also an important defining physical property of a substance, and its measurement can be made with relative ease and modest cost. Tables of the index of refraction for a wide variety of substances, including lipids and oils are readily available for comparison purposes.

The index of refraction for the lipids of the microorganism under examination measured at 1.487 as the average between two different samples. The instrument was calibrated with numerous comparison oil samples and was performing accurately and reliably. The estimated error of the measurement was +/−0.001. The measurement of 1.487 is a relatively high index of refraction, especially as far as oils are concerned. This higher measurement also leads to interpretations of significance.

For example, a relationship exists between the index of refraction and the degree or state of saturation within a fatty acid or lipid. The saturation level (i.e., saturated vs. unsaturated) property of a lipid expresses itself in terms of the bond types within the molecule, an additional aspect of structure.

A saturated fat is one in which a full complement of attached hydrogen atoms exists. A saturated fat contains only single bonds between the carbon atoms. An unsaturated fat, in contrast, has double (or higher) bonds between the carbon atoms, and there will be fewer hydrogen atoms attached as a result. In addition, a distinction may be made between mono-unsaturated fats and poly-unsaturated fats. In essence, a mono-saturated fat has a single double carbon bond within the hydrocarbon chain and a poly-unsaturated fat has more than one double carbon bond within the chain.

The more that can be understood about the structure of a biological molecule, the closer we are towards understanding the behavior, interaction and function of that molecular structure.

A relationship also exists between the degree of saturation in a fat and the ‘iodine number’. The iodine number is a measure of the level of absorption of iodine by fats, and this number can be used in turn to infer the degree of saturation by that same lipid or fat. The method may be used to determine the quality of fats. The degree of fat saturation may affect spoilage rates for food and in turn affects the economics of the food industry. This is an example of an application of embodiments of the present disclosure that extend beyond treatments for Morgellons disease, or the like.

In this study, a relationship was established between the index of refraction of a lipid of the microorganism and the iodine number of that same lipid. An increase in the iodine number is indicative of a higher level of unsaturation and in parallel it has been found that a higher index of refraction is strongly correlated with a higher iodine number. Ultimately, a higher iodine number estimate will indicate a higher level of unsaturation within the lipid.

In this study, several different lipid types were investigated and the correlation between the index of refraction was strong (r=0.92, n=13). The accuracy of the refractometer in use has been included as a part of the study. The result of this work is that a viable method to estimate the level of relative saturation from a direct measurement of the index of refraction of the lipid under study now exists. The application of the linear regression model to the measured index of refraction (1.487) yields an estimate for the iodine value as 218. This magnitude for the estimated iodine value is extremely high and it is significant in its own right.

The conclusion to be reached from this iodine value is meaningful. This study indicates that the character of the lipid may be of a highly poly-unsaturated lipid. This result is corroborative with the interpretation of a relatively lengthy fatty acid chain within the lipid structure. These two interpretations are mutually supportive of one another. The lipid hydrocarbon chains are expected to be lengthy with several double carbon bonds along the chain. This, in turn, affects the structure as double bonds cause a bend to take place in the hydrocarbon chain. Several double bonds enhance that feature further.

In addition, double bonds within a hydrocarbon chain are much more likely to produce chemical reactions. Lipids with a high iodine value are more subject to oxidation and therefore have a greater likelihood of becoming rancid (spoiled). High iodine level lipids are also more likely to produce free radicals. Lastly, highly polyunsaturated lipids are more likely to polymerize (i.e, ‘plasticize’). Each of these impacts may cause additional harm to the body.

There are many health risks associated with polyunsaturated fats. Antioxidants play a role in the mitigation of excessive oxidation to the body, which may be relevant in the amelioration of the harmful influences of polyunsaturated fats. Halogens may have an impact on the thyroid and metabolism.

This study indicates that lipids of the microorganism may be highly subject to the process of oxidation. The character of the lipids is somewhat unusual with respect to oxidation and, for that matter, combustion. The lipids that have been extracted ignite easily. In accordance with exemplary embodiments of the present disclosure, a method involves placing a small amount of the lipids into a watchglass with a small piece of paper acting as a wick. The lipids of the microorganism burned easily and steadily under these conditions, and the behavior is somewhat akin to lamp oil.

Due to the biological and apparent polyunsaturated nature of the lipids, a comparison might be made with whale oil. Fish oils and whale oil share many interesting properties of the highly polyunsaturated fats. In this study, the wick remained at the end of combustion, demonstrating that the oil itself is the primary source of fuel within combustion. This study also demonstrated a failure of any of the other tested lipids or oils to support direct combustion.

Combustion goes hand in hand with oxidation; something that burns oxidizes. In this study, of all the other oils tested under similar conditions (approximately 8 varieties of varying degrees of unsaturation), only the lipids of the microorganism showed any ease of combustion. Along with the highest index of refraction found within the group that has been examined, the dramatic display of combustion of the sample further demonstrates the lipid of the microorganism is highly unsaturated and thus prone to excessive oxidation. This finding corroborates with the excessive oxidation within the body that occurs in association with Morgellons disease or condition.

This study further indicates that excessive oxidation within the body is one of the most likely outcomes expected with the Morgellons disease or condition. Several methods were used to determined excessive oxidation to be involved. One was by observation of the culture growth, whereby the Fe+2 ion is converted to Fe+3 during the growth process. Visibly this is represented by a change in color from a greenish solution (characteristic of Fe+2) to a rust colored solution (characteristic of Fe+3). Also, Fe+3 recovery has taken place directly from the cultures. Data from a study whereby approximately a dozen members of the public, a subset of which made claim to being severely affected by the Morgellons condition, submitted blood image scans for analysis are also indicative of a probable relationship between the state of oxidation of blood (as indicated by color and color spectral analysis) and the severity of Morgellons symptoms reported. In this study, the scanned blood images were processed with a color spectral analysis via NIH software.

There are at least two primary forms of lipids in the body, one for storage of energy within the cells and another within the membranes of the cell, where they act to encapsulate and protect the cell. Saturated fats are more likely to be associated with the storage of energy internal to the cell and unsaturated fats are more likely to be associated with the membranes of a cell. Phospholipids are an important class of lipids that are found within the cell membranes. The degree of unsaturation within phospholipids varies, with one or both tails having double carbon bonds (the site of oxidation).

The oxidation of lipids is referred to as lipid peroxidation, and it is especially prone to occur with polyunsaturated lipids, as in this study. Phospholipids (a bi-layer) are a major constituent of cell membranes, and the oxidation of these lipids subsequently causes damage to the cell. Lipid peroxidation is essentially the theft of electrons from the lipids in the membranes and it occurs as a free radical chain reaction. The oxidation occurs when there is an excess availability of free radicals, or reactive oxygen species. The point of oxidation will be the location of the double bond, which occurs at a location within the unsaturated fatty acid tail.

As shown herein, these microorganisms contain within them a highly polyunsaturated fat and/or fatty acids, that are expected to occur within the membranes of the microorganism. The microorganism may therefore be subject to, or result in, lipid peroxidation in the presence of free radicals. This process, once started, is a chain reaction and is only terminated in the presence of appropriate antioxidants, such as Vitamin E, glutathione peroxidase, transferrin (binding free iron), enzymes (such as catalase), in addition to others. Vitamin C and NAC (N-acetyl cysteine acting as a glutathione precursor) may be effective antioxidants as well.

In this study, a reaction was observed with one of the halogens, in this case, iodine. Similar to the case of combustion, the microorganism lipids under study were the only lipids (of approximately eight in comparison) that displayed a pronounced, and believed to be unique and characteristic, reaction with iodine. Iodine reacts with lipids and this is the very basis of the ‘iodine number’ method used as a measure of the unsaturation level of the lipid. In this study, the formation of a bright red colored iodine complex was observed, which presented itself only within this particular lipid form in relation to numerous sample types that it has been compared with. The colored complex reaction did not occur in like fashion to any other lipid samples examined. The nature of the complex may be of an iron-lipid-iodine or transition metal complex.

Using visible light spectroscopy, it was concluded that the colored complex formed has a structure that contains numerous double carbon bonds. Visible light spectroscopy is highly dependent upon what is termed conjugation; conjugation is a molecular structure that is based upon alternating single and double carbon bonds. The greater the degree of conjugation, the longer the wavelength of the color that will be absorbed. Chromophores are especially likely to form with compounds that involve the transitions metals, such as iron. The color of the complex lends itself well to visual light spectrometry and a spectral plot of the microorganism complex formation.

In this study, the peak absorbance occurred at approximately 498 nanometers. This spectral examination of the lipid-iodine complex is an identification method to establish the presence or existence of this particular microorganism lipid form. The identification of an iron-lipid-iodine complex was further substantiated with tests for the detection of iron using 1,10 phenanthroline reagent in combination with the lipids in a mildly polar solution. These tests were positive for the presence of the Fe+2 ion within the lipids of the microorganism. This finding was in coincidence with significant Fe+2 iron use and metabolism by the microorganism.

A polymer is a molecular structure that is composed of many repeating smaller units. They can be either synthetic or natural, and they usually have a large molecular mass compared to that of the basic structural unit. Latex and styrofoam are examples of both a natural and a synthetic polymer. The architecture and length of the polymer chains strongly affect the physical properties of the polymer, such as elasticity, melting point, and solubility, amongst others.

The reason that polymerization is relevant to this study is that unsaturated lipids are prone to polymerization. The higher the degree of unsaturation, the more likely that polymerization will take place. This is due to the oxidation at the double carbon bonds. A familiar example of polymerization is the use of linseed oil. Linseed oil is a highly unsaturated lipid that is applied to furniture as a protective coating; this is one of the so-called “drying oils”. As this type of oil weathers (or oxidizes), it will form a harder and protective coating over the wood surface. This is an excellent example of the oxidation of a highly unsaturated oil or lipid that produces a polymer. As mentioned, polymers can vary widely in their physical properties, and the plastics are an excellent additional example of synthetic polymers. Oil paints that artists use are another example of the “drying oils” that share these same characteristics.

The probability of polymerization for the lipid complex of the microorganism is believed to be high, as all of the prerequisite characteristics are in place. It may be highly unsaturated and therefore subject to oxidation. As such, the lipids of the microorganism may produce polymers which, in general, would be anticipated to cause harm if internal to the body.

Tests conducted on the microorganism indicate that they are Gram-negative. A Gram-negative test is important for bacteria as it indicates at least three characteristics of importance. These include that the cell walls are lipid-rich in comparison to Gram-positive bacteria; that the negative test indicates the presence of lipopolysaccharides (LPS) or endotoxins within the cell wall; and that the bacteria is likely pathogenic bacteria and associated with endotoxins. A Gram-negative cell is lipid rich, while a Gram-positive cell has a much lower lipid content. The lipid content of the Gram-negative cell wall is approximately 20-30%, which is very high compared to the Gram-positive cell wall.

In this study, the relatively high volume of lipids that were extracted from the microorganism is supportive of the Gram-negative test result.

In the Gram-negative cell, the peptidoglycan layer is about 5-20% by dry weight of the cell wall; in the Gram-positive cell the peptidoglycan layer is about 50-90% of the cell wall by dry weight. Peptidoglycan, also known as murein, is a polymer consisting of amino acids and sugars. Gram negative bacteria are generally more resistant to antibiotics than Gram-negative bacteria. In consideration of the cross-domain terminology currently in use, the archaea can be either Gram-negative or Gram-positive. A difference between the two forms, beyond the relative lipid content and peptidoglycan layer, is the presence of lipopolysaccharides (LPS) on the Gram-negative bacteria. LPS, or endotoxins, elicit a strong immune response in animals.

There are no regulatory standards for the levels of endotoxins in the environment. Endotoxins are associated with increased weight gain, obesity, gum and dental infections and diabetes. A linkage with Chronic Fatigue Syndrome exists, as well as with atherosclerosis, oxidative stress, chronic conditions, cardiovascular disease and Parkinson's Disease. The condition of endotoxins within the blood is referred to as endotoxemia. Many of the above symptoms are also reported in Morgellons disease or condition; this similarity may be indicative of a linkage between Morgellons and endotoxins.

In this study, an infrared investigation into the nature of the extracted lipids was conducted. An IR spectrophotometer was used for this project and a very clear spectrum was obtained. The infrared spectrum is dominated by peaks in the 3400 cm-1, 2900-3050 cm-1, 2000-2100 cm-1, 1450-1650 cm-1, and in the 700 cm-1 region. The primary functional groups under analysis included the alkanes, alkenes, aromatics, alcohols, and thiocyanates. Polymeric phenols and alcohols exist as a primary subject of investigation.

An analysis of the infrared spectrum demonstrates that it is highly dominated by the combination and presence of carbon-carbon and carbon-oxygen single and double bond functional groups. All assessments in this study are highly corroborative of one another and they support the assessment of a highly unsaturated lipid, and all that this entails, as comprising a core structure of the microorganism extraction that has taken place.

Exemplary embodiments of the present disclosure provide a method or methods to isolate and culture this microorganism in both liquid and solid mediums. In exemplary embodiments, the microorganism is isolated and cultured in a highly purified state. By “highly purified” as used herein it is meant that there is no visible contamination by other microorganisms. Some of these methods allow for long term inert storage of the microorganism. The methods are dependent upon establishing a highly acidic growth environment that includes a transition metal complex with additional minimal specific nutrients.

The growth process can be affected or enhanced with additional contributions from oxygen injection and/or direct and alternating current electromagnetic energies. The method allows for sensitivity and/or inhibition of growth testing in both solid and liquid forms. The culture process is scalable and large quantities of the purified organism in the primitive state can be grown if required.

In exemplary embodiments, a method for culturing a microorganism associated with Morgellons disease is provided comprising adding the microorganism to a solution comprising ferrous iron, sugar and water. In some embodiments, growth of the microorganism is primarily coccus form. In some embodiments, the method may also include addition of hydrogen peroxide to the solution. In some embodiments, growth of the microorganism may include coccus form with concomitant filamentous growth. In exemplary embodiments, the method may further comprise exposing the solution to low frequency electromagnetic energy and/or adding compressed air to the solution. In some embodiments, pH of the solution is maintained at about 3.5 to about 5.

In exemplary embodiments, the method may also include assessing growth of the microorganism via microscopic examination, density of the culture, conversion of ferrous iron to ferric iron, infrared spectrophotometry and/or qualitative chemical reactions.

In some embodiments, a composition is provided comprising a microorganism associated with Morgellons disease in a solution comprising ferrous iron, sugar and water. The solution may further comprise hydrogen peroxide and/or maintenance of the pH of the solution at about 3.5 to about 5.

In exemplary embodiments, a method for culturing a microorganism associated with Morgellons disease is provided which may include adding a microorganism to a solid growth medium comprising agar and ferrous iron. The solid growth medium may further comprise sugar, potato broth, ferrous iron mixed with agar, and/or the like. Growth of the microorganism is primarily coccus form. In some embodiments, the solid growth medium may further comprise hydrogen peroxide. In some embodiments, growth of the microorganism may be coccus form with concomitant filamentous growth, or the like. In some embodiments, agar in the solid growth medium may be in the range of about 0.5 to 1.5% solution. In some embodiments, the method may further comprise assessing growth of the microorganism via microscopic examination, density of the culture, conversion of ferrous iron to ferric iron, infrared spectrophotometry and/or qualitative chemical reactions.

In some embodiments, a composition comprising a microorganism associated with Morgellons disease in a solid growth medium comprising agar and ferrous iron is provided. The solid growth medium may further comprise sugar. In some embodiments, the solid growth medium may further comprise hydrogen peroxide. In some embodiments, the agar in the solid growth medium is in the range of about 0.5 to 1.5% solution.

The liquid and solid cultures, when grown in the identified conditions described herein, are highly impervious to contamination from other growth forms. The liquid and solid cultures are amenable to inhibition and sensitivity tests. The acidity of the growth environment is a major factor in attaining successful growth. However, acidity alone is not the only variable that produces the uncontaminated and productive growth.

In some embodiments, drug sensitivity testing may be supported through monitoring of zones of inhibition when the agar format is used. Therefore individual sensitivity testing can be done using this format to determine the best clinical treatment for individual patients. This can also be done serially to determine when organism shedding has been controlled, and if medication regimens need to be changed in response to emerging resistance. Several antibiotics and anti-fungal agents have been screened using this method, none of which have demonstrated visible inhibitory effect upon growth. Specifically, adding Penicillin, Ampicillin, Erythromycin, Neomycin, Tetracycline or Ciprofloxacin to solution had no inhibitory effect on organism growth. In other studies, Itraconazole and Posaconazole were each added to the culture medium followed by the organism. Neither antifungal agent was shown to inhibit growth

In accordance with exemplary embodiments, a method is provided to chemically separate proteins and lipids from a microorganism associated with Morgellons disease. A method may comprise culturing the microorganism in accordance with the methods disclosed herein; preparing a solution comprising the microorganism and bile; blending the bile and microorganism solution with a non-polar solvent; adding an acid and a staining reagent to the solution; (e) blending the bile, microorganism and acid mixture; and (f) separating the solution into lipid and protein layers. Upon separation, the protein layer is a precipitant in the bottom of the solution and the lipid layer is in the upper layer or layers of the solution.

In some embodiments, the method may further comprise isolating a protein or proteins from the protein layer via progressive dilution of extracted precipitant. In some embodiments, the method may comprise adjusting the pH of the extracted precipitant to about 3.5 to about 5.

In exemplary embodiments, the method may further comprise isolating a lipid or lipids from the lipid layer via separation of the lipid layer from the protein layer followed by separation of lipids in the lipid layer from any microorganism residues. A lipid layer may be separated from the protein layer via a separatory funnel.

In some embodiments, lipids in the lipid layer may be separated from any microorganism residues by addition of one or more non-polar solvents, for example, xylene, or the like. In some embodiments, a method for inhibiting growth of a microorganism associated with Morgellons disease may include contacting the microorganism with an inhibitor so that growth of the microorganism is inhibited. Examples of inhibitors include, but are not limited to, antioxidants of n-acetyl cysteine, glutathione, vitamin C and sodium citrate.

The following nonlimiting examples are provided to further illustrate embodiments of the present invention.

### EXAMPLES

### Example 1

### Culturing Methods

The microorganism can be cultivated in a variety of mediums in both liquid and solid form. The solid form of the growth appears to favor both primitive growth and subsequent filament production, whereas the liquid form appears to emphasize sub-micron coccus growth

An embodiment features a liquid environment that comprises a mixture of pure water; a commercial chelated transition-metal complex (Ferti-Lome Liquid Iron) fertilizer solution that is dominated by the presence of ferrous iron, which also includes other metals in small amounts, such as copper, manganese and zinc; fructose; salt; compressed air induced into the solution; incubation with mild heat at approximately 80-120° F., and a combination of Very Low Frequency (VLF) and Extremely Low Frequency (ELF) electromagnetic energy induced into the solution. A further embodiment of the invention includes a liquid environment comprising the following: 500-1500 ml pure water; 15-45 ml commercial chelated transition-metal complex (Ferti-Lome Liquid Iron) fertilizer solution that is dominated by the presence of ferrous iron, and also includes other metals in small amounts, such as copper, manganese and zinc; 10-30 ml fructose; 0.75-2.25 ml salt; compressed air induced into the solution; incubation with mild heat at approximately 80-120° F., and a combination of Very Low Frequency (VLF) and Extremely Low Frequency (ELF) electromagnetic energy induced into the solution. In yet another embodiment, the liquid environment comprises a mixture of 1000 ml pure water; 30 ml of a commercial chelated transition-metal complex (Ferti-Lome Liquid Iron) fertilizer solution that is dominated by the presence of ferrous iron, and also includes other metals in small amounts, such as copper, manganese and zinc; 20 ml fructose; 1.5 ml salt; compressed air induced into the solution; incubation with mild heat at approximately 80-120° F., and a combination of Very Low Frequency (VLF) and Extremely Low Frequency (ELF) electromagnetic energy induced into the solution. Electromagnetic energy (ELF-VLF) may affect the culture growth, but it also may not be required for sufficient or substantial growth to take place. Both the oxygen and the electromagnetic energy additions may comprise ‘variable enhancements’, or the like.

There are numerous variations on the above combination that can also be reasonably successful with growth. For example, a ferrous iron solution (iron sulfate) can be used alone with water, and growth can be produced. Under absolute minimalist conditions, growth has been observed in water samples alone with sufficient time elapsing. The additional factors, such as oxygen and electromagnetic energy variables, listed above appear to enhance growth production but may not necessarily be required. In yet another further embodiment, potato broth may be added to the culture, especially in combination with ferrous iron. The microorganism appears to grow most favorably in an acidic environment, especially in combination with ferrous iron sources. The optimum pH for growth is currently assessed at approximately 3.5 to 5.0, but is preferable at about 4.5. Success of growth can be assessed, in part, by microscopic examination, density of the culture, conversion of ferrous iron to ferric iron, infrared spectrophotometry and qualitative chemical reactions.

Another embodiment of the invention features a solid growth medium to develop growth from the microorganism comprising agar (in the range of 0.5%-1.5% solution, preferably as a 1% solution), potato broth, commercial chelated transition metal complex (Ferti-Lome), fructose and salt. In a further embodiment, the solid growth medium comprises 50-150 ml agar (in the range of 0.5%-1.5% solution, preferably as a 1% solution), 5-15 ml potato broth, 2.5-7.5 ml commercial chelated transition metal complex (Ferti-Lome), 1.5-4.5 ml fructose and 0.15-0.45 ml salt. In yet another embodiment, the solid growth medium comprises 100 ml agar (in a 1% solution), 10 ml potato broth, 5 ml commercial chelated transition metal complex (Ferti-Lome), 3 ml fructose and 0.3 ml salt. Such mixture then undergoes incubation with mild heat at a range of 80-120° F.

In another embodiment, it has been shown that incubation of the solution is not required. Increased heat does, however, appear to help restrict growth to the “coccus” form. There are two primary forms of growth that develop, coccus and filamentous. Coccus always occurs first, filamentous can develop depending on the culture medium is. An embodiment of a growth medium occurring at room temperature for growth of primarily coccus form comprises the microorganism, 30 ml water, 0.10 grams ferrous sulfate and 0.25 grams sugar (table sugar or fructose are equally effective). An embodiment of a growth medium occurring at room temperature for growth of coccus form with subsequent concomitant filamentous growth comprises the microorganism, 30 ml water, 0.10 grams ferrous sulfate, 0.25 grams sugar (table sugar, fructose are equally effective) and 6 drops 3% hydrogen peroxide.

After a period of incubation and being subjected to the above conditions and environment, the organism will exist in a primitive state in relatively large numbers and purified form. The microorganism can be further isolated by gravity separation from the growth medium solution and subsequently rinsed in pure water. This process can be repeated until only concentrated microorganism growth remains in an aqueous solution. The microorganism appears to be inert, storable and transportable at room temperature under these final conditions of isolation and purification for a relatively long period of time (several weeks at a minimum). A dried form of storage has also proved successful in sustaining the viability of growth.

### Example 2

### Isolation of Proteins and Lipids

Other embodiments of the invention disclose methods to chemically isolate protein from this microorganism, which can include a multi-stage process that involves the use of bile salts, alkaline solutions, incubation, non-polar solvents, agitation, acidification, gravity separations, dilution, the addition of a transition metal complex, additional alkalizing agents and/or precipitation. The resulting protein is chemically inert and can be placed in relatively long term storage.

An embodiment of the invention relates to a multi-step method to chemically isolate protein from the microorganism and may comprise, for example:
1. Development of the microorganism culture;
2. Preparation of a bile-microorganism solution comprising:
   a. 550 ml H₂O heated to approximately 40° C.
   b. 20 ml. Ox-Bile Powder or Ox-Bile salt equivalent (e.g. NutnCology Ox Bile 500 mg. capsules);
   c. 80 ml. purified, concentrated, gravity-settled microorganism within water; and
   d. Sodium hydroxide (NaOH).
3. Mixing and blending of the bile-microorganism solution with a non-polar solvent such as xylene, in a high level of agitation for approximately five minutes:
4. The addition of an acid and a reagent to the solution;
5. Blending of the bile-microorganism-acid complex mixture under high agitation for approximately five minutes; and
6. Separation of the solution into the lipid and protein layers, in which the protein layer is in the bottom of the solution, and the lipid layer is in the upper layer or layers of the solution.

The process and preparation of the bile-microorganism solution in Step (2) is scalable and can be produced in both small and large quantities. The sodium hydroxide (NaOH) is added to solution to bring the pH of the bile-microorganism solution to approximately 9.0 to 9.5 on the pH scale. This solution is incubated at a temperature of 85-95° F. until the pH of the solution drops to approximately 6.5-7.5. This process also produces the release of fatty acids into the solution. The time for this process to occur is usually between 3 to 5 days. The reduced pH bile-microorganism solution is subsequently mixed with the solvent xylene. The proportions are approximately four parts bile-microorganism solution to one part xylene. The solution is then blended with a high level of agitation for approximately five minutes. The solution will change in color from dark brown to a milky tan color when the agitation is completed.

When adding an acid and a reagent to the solution in Step (4), the proportion is 0.5-1.5 ml. of 8.7M hydrochloric acid (HCl) per 100 ml. of bile-microorganism solution. In addition, approximately 1-3 ml. of a staining reagent such as Bradford reagent can be added per 100 ml. of bile-microorganism solution. The Bradford reagent under usage can be made according to the following custom proportions:
a). 15 ml. Coomassie Protein Solution, Carolina Biological Supply, stock number 21-9784;
b). 20 ml. Phosphoric Acid;
c). 10 ml. H2O;
d). 5 ml. ethanol

When the bile-microorganism-acid complex mixture is blended again under high agitation for approximately five minutes in Step (5), the solution will turn lighter in color when this process is completed.

To separate the solution in Step (6), the blended bile-microorganism-acid complex solution is placed into a separatory funnel to separate the protein and lipid layers. Two distinct layers will be observed to form over time; a dark thin layer at the top and a lighter color solution in the majority at the bottom of the funnel. The amount of blending will affect the layer formation process and less thorough blends will affect the layered development that occurs. In all cases, isolated protein is observed in the bottom layer of any that occur within the separatory funnel; it will appear as milky in color and constitution. The lowest layer within the separatory funnel is extracted

Embodiments of the present disclosure also may include a further protein extraction process, in which the precipitant at the bottom of the separatory funnel can be further extracted if needed. Make at 50%/50% solution of the extracted lower layer with an equal proportion of acetone (ml per ml). This solution can be gravitationally separated. If necessary this same process can be additionally repeated to the desired level of purification.

Embodiments of the present disclosure also may include a dilution-precipitation method for protein isolation, which may be a multistep method to chemically isolate protein from the coccus organism can occur with progressive dilution of extracted precipitant from prior steps 1 thru 6.

The secondary extracted and separated protein rich layer from the bile-microorganism-xylene-acid-non-polar-solvent complex is diluted with water in a ratio of 10 to 15 parts of water to 1 part extraction layer. The first is the addition of a ferrous iron compound to the dilute solution.

Without being limited to any particular theory, a critical and limiting reagent appears to be that of the ferrous iron ion. Ferrous sulfate (1M concentration) is added to the dilute solution on the ratio of 2 ml. per 100 ml. of dilute solution. A commercial ferrous iron complex (Ferti-Lome Chelated Liquid Iron) can also be used. This is a chelated fertilizer solution that is high in the concentration of ferrous iron with the addition of certain trace minerals and metals.

The second part of the precipitation process is the altering of the pH of the dilute solution. In combination with the addition of the ferrous iron complex (this complex also affects the pH of the solution and makes it more acidic), the pH of the solution is slowly and carefully made more alkaline with the addition of sodium hydroxide (NaOH). It is presumed that any strong alkaline agent, such as potassium hydroxide (KOH) could be used with equal success. The pH of the dilute extraction lower is quite low, on the order of 1 to 2, and is therefore highly acidic in nature. The optimum production of precipitate appears to occur at a pH of approximately 3.5 to 4.0. The color of the precipitate formed can vary from off-white to tan to green to dark green depending upon the final pH achieved. The density of the precipitate can vary to some degree in both density and color and it may depend upon the variation of ferrous iron complex that is used to alkalize the solution and final pH.

Existence of the precipitate as a protein complex can be shown by the following three exemplary methods. The first of these is visual analysis; the protein precipitate derives from a solution that is primarily milky white in nature; it has a consistency similar to many proteins that are soluble. The second is the Bradford reagent test; this test occurs under acidic conditions and produces a rich blue complex that can also be examined spectroscopically (595 nanometers). The Biuret test for proteins has been determined to be much less reliable because of reagent stability issues; the Bradford reagent test is reliable and has been subjected to numerous control protein solutions. Acidification of the precipitate complex appears to facilitate breakdown and solubility of the protein precipitate complex. The third method used is ultraviolet detection; the absorbance ratio of protein solutions formed from 260 and 280 nanometer observation is commonly used to estimate concentrations of general protein solutions. The protein concentration of 10 to 30 mg. per ml. of solution has been observed in the test above. Long term storage in both liquid and dried forms may maintain the integrity of the protein complex.

An embodiment of the invention discloses a multi-step method to chemically isolate lipids from the microorganism may comprise, for example:
1. Development of the microorganism culture;
2. Preparation of a bile-microorganism solution comprising:
   a. 550 ml H₂O heated to approximately 40° C.
   b. 20 ml. Ox-Bile Powder or Ox-Bile salt equivalent (e.g. NutnCology Ox Bile 500 mg. capsules);
   c. 80 ml. purified, concentrated, gravity-settled microorganism within water; and
   d. Sodium hydroxide (NaOH).
3. Mixing and blending of the bile-microorganism solution with a non-polar solvent such as xylene lightly for a short period of time.
4. The addition of an acid and a reagent to the solution; when adding an acid and a reagent to the solution in Step (4), the proportion is 0.5-1.5 ml. of 8.7M hydrochloric acid (HCl) per 100 ml. of bile-microorganism solution. In addition, approximately 1-3 ml. of a staining reagent such as Bradford reagent can be added per 100 ml. of bile-microorganism solution. The Bradford reagent under usage can be made according to the following custom proportions:
   a). 15 ml. Coomassie Protein Solution, Carolina Biological Supply, stock number 21-9784;
   b). 20 ml. Phosphoric Acid;
   c). 10 ml. H₂O;
   d) 5 ml. ethanol.
5. Blending, mixing or shaking of the bile-microorganism-acid complex mixture very lightly for a short period of time.
6. Separation of the solution into the lipid and protein layers, in which the protein layer is in the bottom of the solution, and the lipid layer is in the upper layer or layers of the solution.

To separate the solution in Step (6), the blended bile-microorganism-acid complex solution is placed into a separatory funnel to separate the protein and lipid layers. Lipid isolation is in the upper layer or layers of any that occur within the separatory funnel. These will be found to be lipid rich layers within the solution.

If the extracted upper layer as described above is subsequently mixed with an equal volume of non-polar solvent such as acetone, and shaken briskly and then placed into a separatory funnel, further separation will take place. The solution will be comprised of two main portions. One layer, darker in color and in the minority, will be comprised of microorganism residues. The other layer will be a non-polar mixture comprised predominantly of xylene, a non-polar solvent (such as acetone) and lipids. The xylene, non-polar solvent (such as acetone) and lipid layer is separated from the mixture. If this mixture is allowed to settle further (or centrifuged), a lipid layer (in the minority) will rest on the top of the solution and the xylene-acetone mix will lie below. The lipid layer will have an oily and clear appearance. The lipid layer is then separated from the mixture.

If the lipid layer is placed into water, it will be found that it is completely insoluble. The two layers can be mixed and they will return to a state of separation, either by centrifugation or gravity settling over time. The insoluble nature of the layer meets the primary definition of a lipid, in this case extracted from the microorganism. The lipids can be stored in this inert state for relatively long periods of time (several weeks minimum) either combined with water or alone. They can also be transported safely in this state as well.

The visible and insoluble properties establish the existence of a lipid derived from the microorganism. Furthermore, the lipids can be subjected to an emulsion test with a combination of water and ethanol (or other alcohol). It will pass that test and turn whitish as an oil emulsion. In addition, the lipids can be examined under the microscope and they satisfy all expected appearances of lipid or fat concentrations.

In yet another further embodiment, the multi-step method to chemically isolate lipids from the microorganism further comprises the step extracting the upper layer of the separated solution with equal portions of acetone. If this mixture is placed into a separatory funnel, a further separation will take place. Of note, the upper layers of this separation process contain lipids. The process of secondary separation can be repeated to the desired level of purification.

While the foregoing is directed to embodiments of the present invention, other and further embodiments of the invention may be devised without departing from the basic scope thereof. It is also understood that various embodiments described herein may be utilized in combination with any other embodiment described, without departing from the scope contained herein.

What is claimed is:

1. A method for culturing a microorganism associated with Morgellons disease, said method comprising adding the microorganism to a solution comprising ferrous iron, sugar and water.

2. The method of claim 1 wherein growth of the microorganism is primarily coccus form.

3. The method of claim 1 further comprising adding hydrogen peroxide to the solution.

4. The method of claim 3 wherein growth of the microorganism is coccus form with concomitant filamentous growth.

5. The method of claim 1 further comprising exposing the solution to low frequency electromagnetic energy.

6. The method of claim 1 further comprising adding compressed air to the solution.

7. The method of claim 1 wherein pH of the solution is maintained at about 3.5 to about 5.

8. The method of claim 1 further comprising assessing growth of the microorganism via microscopic examination, density of the culture, conversion of ferrous iron to ferric iron, infrared spectrophotometry and/or qualitative chemical reactions.

9. A composition comprising a microorganism associated with Morgellons disease in a solution comprising ferrous iron, sugar and water.

10. The composition of claim 9 wherein the solution further comprises hydrogen peroxide.

11. The composition of claim 9 wherein pH of the solution is maintained at about 3.5 to about 5.

12. A method for culturing a microorganism associated with Morgellons disease, said method comprising adding the microorganism to a solid growth medium comprising agar and ferrous iron.

13. The method of claim 12 wherein the solid growth medium further comprises sugar, potato broth, or ferrous iron mixed with agar.

14. The method of claim 13 wherein growth of the microorganism is primarily coccus form.

15. The method of claim 13 wherein the solid growth medium further comprises hydrogen peroxide.

16. The method of claim 15 wherein growth of the microorganism is coccus form with concomitant filamentous growth.

17. The method of claim 12 wherein the agar in the solid growth medium is in the range of about 0.5 to 1.5% solution.

18. The method of claim 12 further comprising assessing growth of the microorganism via microscopic examination, density of the culture, conversion of ferrous iron to ferric iron, infrared spectrophotometry and/or qualitative chemical reactions.

19. A composition comprising a microorganism associated with Morgellons disease in a solid growth medium comprising agar and ferrous iron.

20. The composition of claim 19 wherein the solid growth medium further comprises sugar.

21. The composition of claim 20 wherein the solid growth medium further comprises hydrogen peroxide.

22. The composition of claim 19 wherein the agar in the solid growth medium is in the range of about 0.5 to 1.5% solution.

23. A method to chemically separate proteins and lipids from a microorganism associated with Morgellons disease, said method comprising:
(a) culturing the microorganism in accordance with the method of any of claims 1 through 8;
(b) preparing a solution comprising the microorganism and bile;
(c) blending the bile and microorganism solution of step (b) with a non-polar solvent;
(d) adding an acid and a staining reagent to the solution of step (c);
(e) blending the bile, microorganism and acid mixture of step (d); and
(f) separating the solution of step (e) into lipid and protein layers, wherein the protein layer is a precipitant in the bottom of the solution and the lipid layer is in the upper layer or layers of the solution.

24. The method of claim 23 further comprising isolating a protein or proteins from the protein layer of step (f) via progressive dilution of extracted precipitant.

25. The method of claim 24 further comprising adjusting the pH of the extracted precipitant to about 3.5 to about 5.

26. The method of claim 23 further comprising isolating a lipid or lipids from the lipid layer of step (f) via separation of the lipid layer from the protein layer followed by separation of lipids in the lipid layer from any microorganism residues.

27. The method of claim 26 wherein the lipid layer is separated from the protein layer via a separatory funnel.

28. The method of claim 27 wherein lipids in the lipid layer are separated from the microorganism residues by addition of one or more non-polar solvents.

29. The method of claim 27 wherein the non-polar solvent is xylene.

30. A method for inhibiting growth of a microorganism associated with Morgellons disease, said method comprising contacting the microorganism with an inhibitor selected from the group consisting of an antioxidant of n-acetyl cysteine, glutathione, vitamin C and sodium citrate so that growth of the microorganism is inhibited.
